(19) United States
(12) Patent Application Publication
(10) Pub. No.: US 2026/0241220 A1
(43) Pub. Date: Aug. 20, 2026

(54) WEIGHT PLATE WITH EMBEDDED COOLING COMPONENT

(71) Applicants: Michael Tanner, Woodbine, GA (US); David Loveday, Woodbine, GA (US); Katie Tanner, Woodbine, GA (US); Sophia Loveday, Woodbine, GA (US)

(72) Inventors: Michael Tanner, Woodbine, GA (US); David Loveday, Woodbine, GA (US); Katie Tanner, Woodbine, GA (US); Sophia Loveday, Woodbine, GA (US)

(21) Appl. No.: 19/531,682

(22) Filed: Feb. 6, 2026

Related U.S. Application Data
(60) Provisional application No. 63/760,575, filed on Feb. 19, 2025.

(51) Int. Cl. A63B 21/072; A61F 7/02; A61F 7/10; A63B 21/00; A63B 71/00

(52) U.S. Cl. CPC A63B 21/072; A61F 7/103; A63B 21/4035; A63B 71/0036; A61F 2007/0219; A63B 2071/009; A63B 2225/66

(57) ABSTRACT

A weight plate with a cooling component integrated therein is disclosed. The weight plate includes a plate made of metal and having a hollow portion forming a cavity. A cold pack is positioned in the cavity. The weight plate further includes a handle for gripping the plate. A fill port is integrally formed in the hollow portion of the plate and is in fluid communication with the cold pack.

### CROSS-REFERENCE TO RELATED APPLICATIONS

This application claims priority under 35 USC § 119(e) of U.S. Provisional Application Ser. No. 63/760,575, filed on Feb. 19, 2025, and entitled “WEIGHT PLATE WITH EMBEDDED COOLING COMPONENT,” the entire disclosure of which is incorporated herein by reference.

### TECHNICAL FIELD

The present invention is generally directed to strength training, and more particularly to a weight plate embedded with a cooling component, enabling physical conditioning for low-impact exercises while also maintaining a normal core temperature of the body during training.

### BACKGROUND OF THE INVENTION

Rucking is the action of walking and/or jogging with added weight (e.g., sand, books, weight plates, dumbbells, etc.) inside of a backpack on your back and/or your front across the chest and stomach areas. Walking with a weighted rucksack or backpack is a low-impact exercise, and carrying weight is a normal part of a person's daily routine. Rucking is used commonly by fitness enthusiasts, in military training, and as physical conditioning in police forces.

Athletes training or competing outdoors can be vulnerable to heat stress, which can result in illnesses such as heat cramps, heat exhaustion, dehydration, fainting, and heat stroke. All of these illnesses can have a major impact on both performance and health, especially when hiking or trekking with a heavy backpack in the hot summer, up steep trails. or in deserts.

Thus far, solutions have focused on adding portable ice packs to the surface of the body or to an inside pocket of the backpack/rucksack or piece of garment, or carrying extra water in a water bottle or via a backpack mounted water bladder with a hose for drinking the water. These methods have significant shortcomings. Portable ice packs require carrying an extra piece of equipment with you along with the weighted materials and adding another piece of insulation between the cold pack and the skin. While water bottles and hydration bladders help with hydration, they do not provide direct cooling over a thermal region of the body, eventually run out of water during use, and cannot be refilled on the trail. They are also bulky and water bladders in particular may require emptying half the backpack to access the bladder.

What is needed is a single, integrated weight plate with embedded cooling component that provides the benefits of resistance training while also protecting against heat stress.

### SUMMARY OF THE INVENTION

In accordance with one embodiment of the present invention, a weight plate with a cooling component integrated therein is disclosed. The weight plate further includes a plate made of metal and a hollow portion forming a cavity therein. A cold pack is positioned embedded or situated-in the cavity. The weight plate also include a first handle for gripping the plate.

The weight plate may have an optional fill port integrally formed in one section of the plate and in fluid communication with the cold pack. In this optional embodiment, the fill port is sealed closed with a cap. In other embodiments, with no fill port formed in the plate, the cold pack is either sealed closed with a cap or is completely scaled on all sides and not fillable with water or any other substance.

In some embodiments, the cold pack is encapsulated in the cavity of the plate.

The cold pack may be, but is not limited to, an ice pack or a gel pack. In some embodiments, the cold pack contains a non-toxic blue liquid that freezes colder than water.

In some embodiments, the plate may be placed in the freezer overnight to solidify the cold pack before use or after use. In other embodiments, the plate is placed in the freezer for a sufficient period of time for the cold pack to solidify before use

The weight plate may further comprise a protective wrap positioned over one or more end portions or edges of the plate. In one embodiment, the protective wrap is a soft neoprene type material.

The first handle may be either a hand hold grip or simply an opening or aperture. In some embodiments, the first handle is located between a proximal end of the cavity and a proximal end of the plate. The proximal end of the plate may be adjacent to the fill port. In some embodiments, the fill port may be located at a proximal end of the end or, in another embodiment, at a location anywhere between the proximal end and the distal end of the plate.

In some embodiments, the first handle is an elongated, ergonomically shaped handle with round edges. The first handle may be, but is not limited to, about two inches tall and about four and a half inches wide.

The weight plate may further comprise a second handle, similar in shape and size to the first handle. The second handle may be located opposite the first handle. In some embodiments, the second handle is located between a distal end of the cavity and a distal end of the plate.

The plate may weigh a variety of weights. In some embodiments, the plate is between about one pound to about fifty pounds. In other embodiments, the plate is between about five pounds to about thirty pounds.

The plate may be carried in a rucksack, a backpack, or a vest against a user's body in a packing mode.

The plate may have a variety of widths, lengths (or heights), and thicknesses. In some embodiments, the plate has an overall width between about five inches to twelve inches, an overall length or height between about five inches to twenty inches, and an overall thickness between about one-half inch to about one and a half inches. In some embodiments, the plate has a height or length, width, and thickness that is selected so that the plate slidingly fits within the vest or a pocket of a backpack or rucksack without substantial movement therein.

The cavity, handle(s), and fill port may be created using manufacturing techniques known in the art.

In another embodiment of the present invention, a weight plate with a cooling component integrated therein is disclosed. The weight plate includes a plate made of metal and has a hollow portion forming a cavity. The weight plate also include a cold pack positioned in the cavity.

The weight plate may have an optional fill port integrally formed in one section of the plate and in fluid communication with the cold pack. In this optional embodiment, the fill port is sealed closed with a cap. In other embodiments, with no fill port formed in the plate, the cold pack is either sealed closed with a cap or is completely sealed on all sides and not fillable with water or any other substance.

In some embodiments, the cold pack is encapsulated in the cavity of the plate.

The cold pack may be, but is not limited to, an ice pack or a gel pack. In some embodiments, the cold pack contains a non-toxic blue liquid that freezes colder than water.

The weight plate may further comprise a protective wrap positioned over one or more end portions or edges of the plate. In one embodiment, the protective wrap is a soft neoprene type material.

The plate may weigh a variety of weights. In some embodiments, the plate is between about one pound to about fifty pounds. In other embodiments, the plate is between about five pounds to about thirty pounds.

The plate may be carried in a rucksack, a backpack, or a est against a user's body in a packing mode.

In some embodiments, the plate has an overall width between about five inches to twelve inches, an overall length or height between about five inches to twenty inches, and an overall thickness between about one-half inch to about one and a half inches.

### BRIEF DESCRIPTION OF THE DRAWINGS

FIG. 1A is a front perspective view of a weight plate with a cooling component integrated therein, in accordance with one embodiment of the present invention.

FIG. 1B is a front perspective view of a weight plate with a cooling component integrated therein, accordance with one embodiment of the present invention.

FIG. 1C is a front perspective view of a weight plate with a cooling component integrated therein, accordance with one embodiment of the present invention.

FIG. 2A is a front perspective view of a weight plate with a cooling component integrated therein, in accordance with one embodiment of the present invention.

FIG. 2B is a front perspective of a weight plate with a cooling component integrated therein, in accordance with one embodiment of the present invention.

FIG. 2C is a front perspective of a weight plate with a cooling component integrated therein, in accordance with one embodiment of the present invention.

FIG. 3A is a handle for gripping the weight plate, in accordance with one embodiment of the present invention.

FIG. 3B is a front perspective of a weight plate with a cooling component integrated therein, in accordance with one embodiment of the present invention. In this one embodiment for a five pound to ten pound plate, the length/height may be approximately six inches, the width may be approximately nine inches, and thickness may be approximately one inch.

FIG. 3C is a front perspective of a weight plate with a cooling component integrated therein, in accordance with one embodiment of the present invention. In this one embodiment for a fifteen pound to twenty pound plate, the length/height may be approximately fifteen inches, the width may be approximately seven inches, and thickness may be approximately one inch.

FIG. 3D is a front perspective of a weight plate with a cooling component integrated therein, in accordance with one embodiment of the present invention. In this one embodiment for a thirty pound to thirty five pound plate, the length/height may be approximately fifteen inches, the width may be approximately ten inches, and thickness may be approximately one inch.

FIG. 3E is a front perspective of a weight plate with a cooling component integrated therein, designed to fit or slide in the vest or pocket of backpack or rucksack, in accordance with one embodiment of the present invention.

### DETAILED DESCRIPTION OF THE PREFERRED EMBODIMENTS

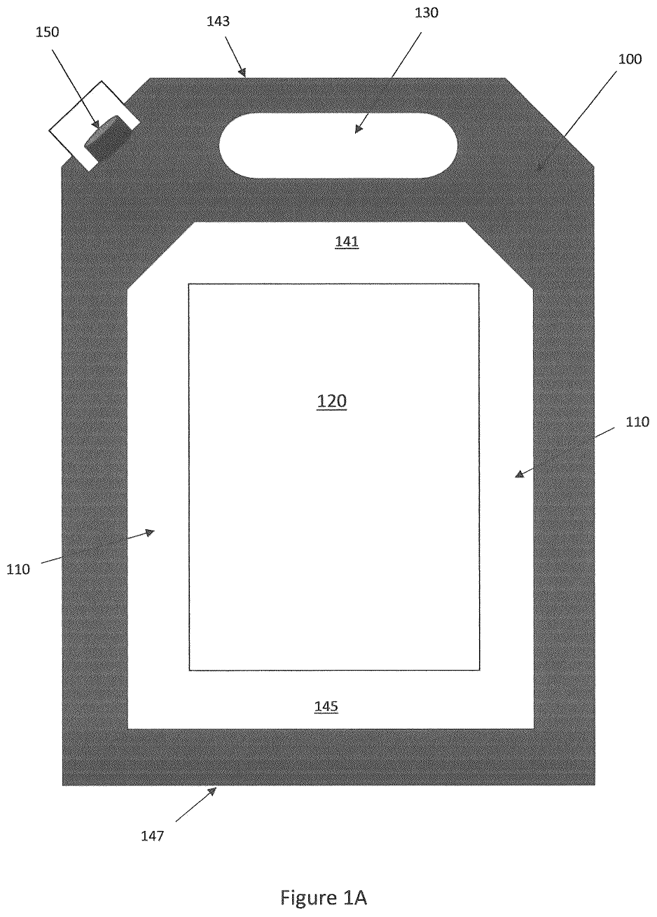

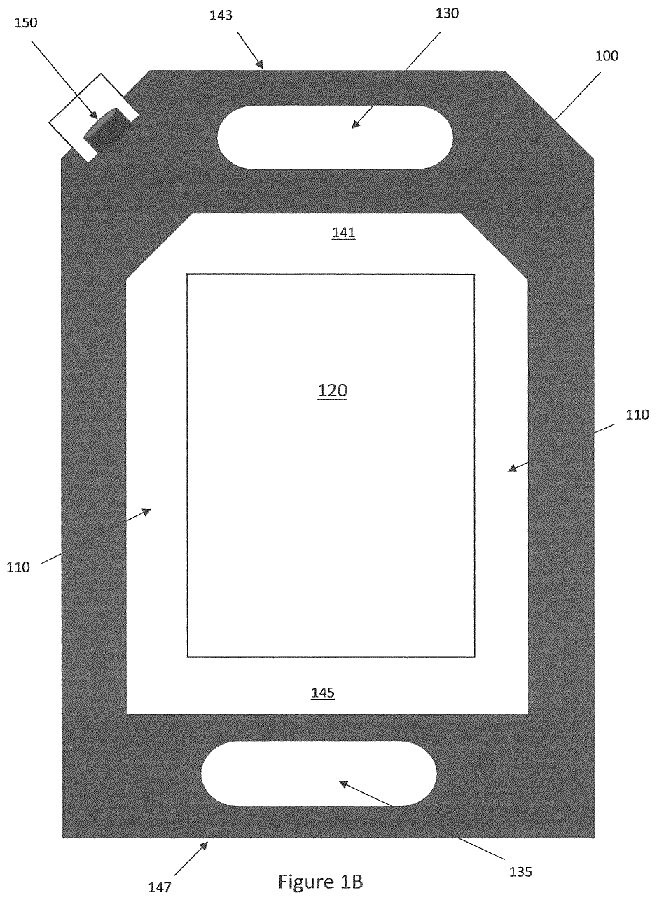

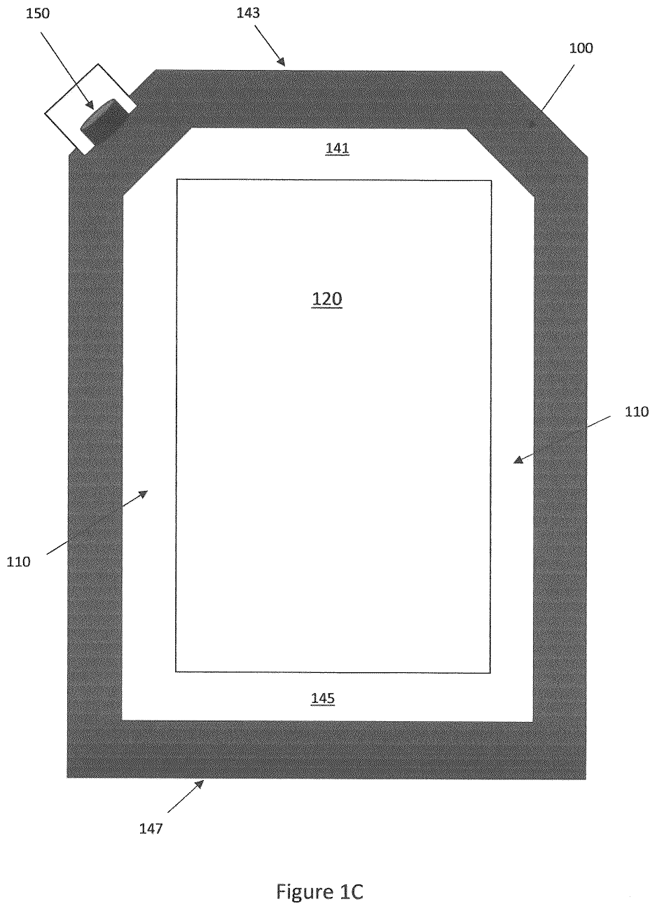

FIG. 1A is a front perspective view of a weight plate with a cooling component integrated therein, in accordance with one embodiment of the present invention. The weight plate includes a plate 100 made of metal with a hollow portion forming a cavity 110. A cold pack 120 is embedded or situated in the cavity 110. In one embodiment, the cold pack 120 is encapsulated in the cavity 110. The plate 100 includes a handle 130 for gripping the plate 100 and may be ergonomically shaped with rounded edges. In FIG. 1A, the handle 130 is shown as a hollow, elongated grip handle, which is an opening formed in the body of the plate 100, between a proximal end 141 of the cavity 110 and a proximal end 143 of the plate 100. It should be noted that the elongated grip handle 130 can be formed at other locations on the plate around the cavity 110, such as between a distal end 145 of the cavity 110 and a distal end 147 of the plate 100, and is not limited to being formed near the proximal end 143 of the plate 100.

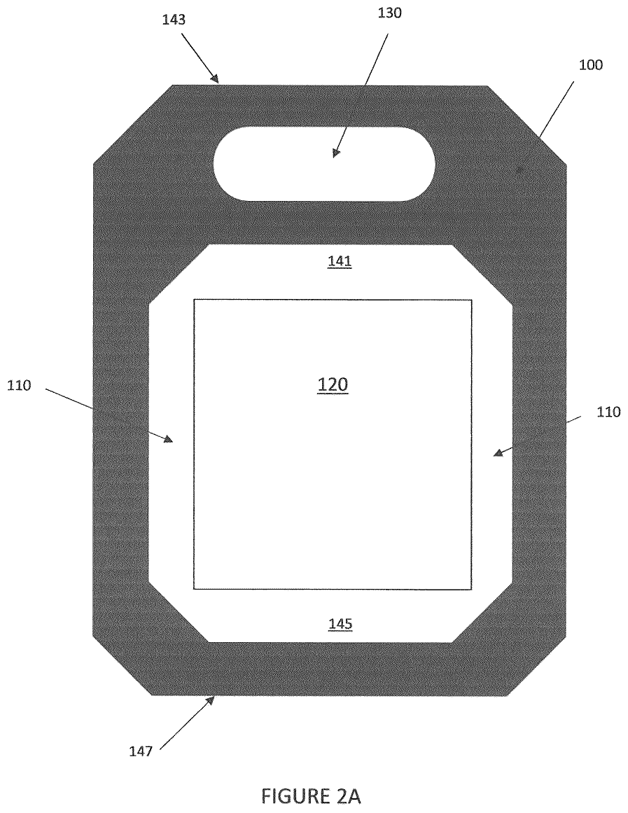

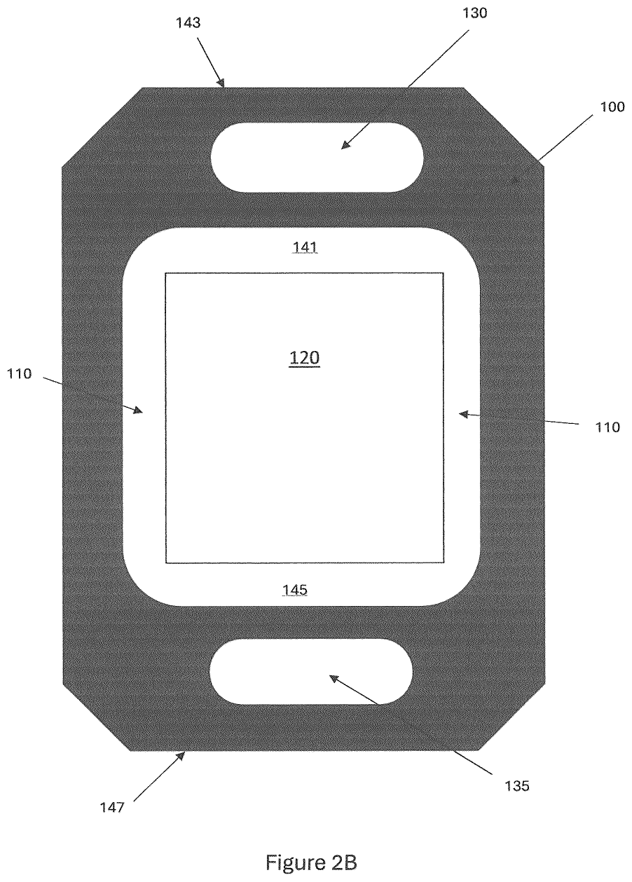

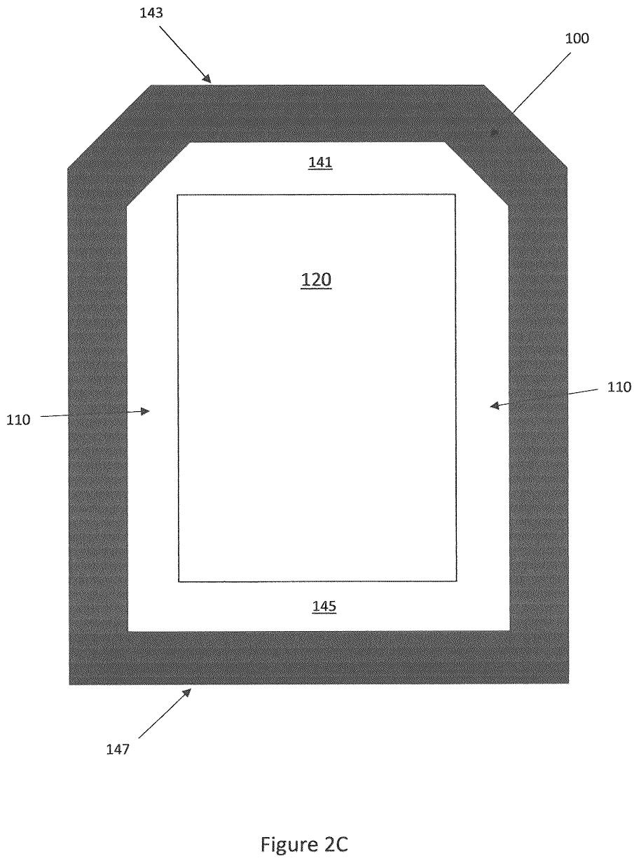

Still referring to FIG. 1A, an optional fill port 150 is shown disposed along a corner of the upper side near the proximal end 143 of the plate 100. The fill port 150 is. integrally formed in the hollow portion forming a cavity 110 of the plate 100 and is in fluid communication with a fluid passageway to the cold pack 120. The fill port 150 is sealed closed with a cap, which may be removable. In other embodiments, as will be discussed in connection with FIGS. 2A-2C, the weight plate has no fill port 150 and the cold pack 120 is sealed tight with a cap or is completely sealed along all sides of the cold pack 120.

The cold pack 120 may be, but is not limited to, an ice pack or a gel pack. In some embodiments, the cold pack 120 contains a non-toxic blue liquid that freezes colder than water. It should be noted that any temperature-responsive liquid may be present in or added to the cold pack 120 which substantially freezes to a solid state when cooled to a suitable temperature, such as by placing the cold pack 120 in a conventional residential freezer until ready for use.

The weight plate may also include an optional protective wrap positioned over one or more end portions or edges of the plate 100. In one embodiment, the protective wrap is a soft neoprene type material.

The plate 100 can weigh anywhere between one to sixty pounds or more. In other embodiments, the plate 100 weighs at least five pounds. The plate 100 may have an overall width between about five inches to twelve inches, an overall height (or length) between about five inches and about twenty inches, and an overall thickness between about one half inch to one and a half inches. Any sized or shaped plate 100 that is suitable to be carried in a rucksack, backpack, or vest against a user's body in a packing mode is suitable for different embodiments of the present invention. In some embodiments, the plate 100 has a height, width, and thickness selected so that the plate 100 slidingly fits within the vest or a pocket of a backpack or rucksack without substantial movement therein.

The cavity 110, handle 130, and optional fill port 150 may be created using any manufacturing technique known in the art.

FIG. 1B is a front perspective view of a weight plate with a cooling component integrated therein, accordance with one embodiment of the present invention. The difference between FIG. 1B and Figure IA is that the weight plate in FIG. 1B has two handles 130 and 135. In the embodiment of FIG. 1B, a first handle 130 for gripping is formed between a proximal end 141 of the cavity 110 and a proximal end 143 of the plate 100 adjacent to the fill port 150, and a second handle 135 for gripping is formed between a distal end 145 of the cavity 110 and distal end 147 of the plate 100. It should be noted that the two handles 130 and 135 may be formed at alternative locations in the plate body. For example, the first handle 130 can be located on one side of the plate 100 between the proximal end 141 and distal end 145 of the cavity 110, and the second handle 135 can be formed on the side opposite to the first handle 135 when the first handle is formed between the proximal end 141 and distal end 145 of the cavity 110.

FIG. 1C is a front perspective view of a weight plate with a cooling component integrated therein, accordance with one embodiment of the present invention. The difference between FIG. 1C and FIG. 1A and 1B is that the weight plate of FIG. 1C has no handle. The weight plate includes a plate 100, a hollow portion forming a cavity 110, a cold pack 120, and a fill port 150 integrally formed in the hollow portion forming a cavity 110 of the plate 100. A proximal end 141 of the cavity 110, a distal end 145 of the cavity 110, a proximal end 143 of the plate 100, and a distal end 147 of the plate 100 are also shown.

FIG. 2A is a front perspective view of a weight plate with a cooling component integrated therein, in accordance with one embodiment of the present invention. FIG. 2A is similar to FIG. 1A but with one difference. The weight plate of FIG. 2A has no fill port. The weight plate includes a plate 100, a hollow portion forming a cavity 110, a cold pack 120, a handle 130 between a proximal end 141 of the cavity 110 and a proximal end 143 of the plate 100. It should be noted that the handle 130 can be formed at other locations on the plate around the cavity 110, such as between a distal end 145 of the cavity 110 and a distal end 147 of the plate 100, and is not limited to being formed near the proximal end 143 of the plate 100. In the embodiment of FIG. 2A, the cold pack 120 is either sealed closed with a cap or is completely sealed on all sides and not fillable with water or any other substance.

FIG. 2B is a front perspective of a weight plate with a cooling component integrated therein, in accordance with one embodiment of the present invention. FIG. 2B is similar to FIG. 1B but with one difference. The weight plate of FIG. 2B bas no fill port. The weight plate includes a plate 100, a hollow portion forming a cavity 110, and a cold pack 120. A first handle 130 for gripping is formed between a proximal end 141 of the cavity 110 and a proximal end 143 of the plate 100, and a second handle 135 for gripping is formed between a distal end 145 of the cavity 110 and distal end 147 of the plate 100. It should be noted that the two handles 130 and 135 may be formed at alternative locations in the plate body. For example, the first handle 130 can be located on one side of the plate 100 between the proximal end 141 and distal end 145 of the cavity 110, and the second handle 135 can be formed on the side opposite to the first handle 135 when the first handle is formed between the proximal end 141 and distal end 145 of the cavity, In the embodiment of FIG. 2B, the cold pack 120 is either scaled closed with a cap or is completely sealed on all sides and not fillable with water or any other substance.

FIG. 2C is a front perspective of a weight plate with a cooling component integrated therein, in accordance with one embodiment of the present invention. FIG. 2C is similar to FIG. 1C but with one difference. The weight plate of FIG. 2C has no fill port. The weight plate includes a plate 100, a hollow portion forming a cavity 110, a cold pack 120, a proximal end 141 of the cavity 110, a proximal end 143 of the plate 100, a distal end 147 of the plate 100, and a distal end 145 of the cavity 110. In the embodiment of FIG. 2C, the cold pack 120 is either scaled closed with a cap or is completely sealed on all sides and not fillable with water or any other substance.

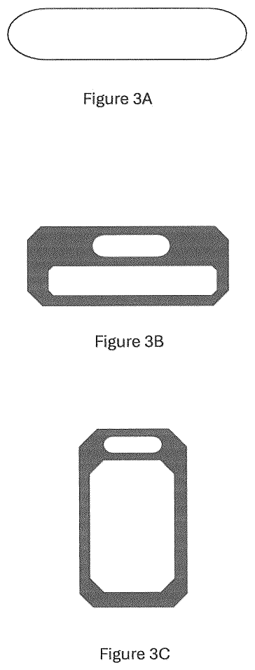

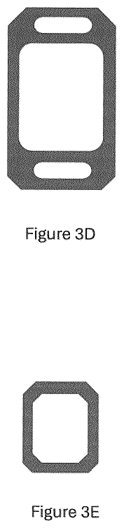

FIG. 3A is a handle for gripping the weight plate as depicted in the previous figures—FIG. 1A, FIG. 1B, FIG. 2A, and FIG. 2B—in accordance with one embodiment of the present invention. In the embodiment of FIG. 3A, the handle is approximately two inches in height and four and a half inches in width. It should be noted that a plurality of other sizes and shapes can be used for the handle.

FIG. 3B is a front perspective of a weight plate with a cooling component integrated therein, similar to the one or single handle weight plates depicted in FIG. 1A and FIG. 2A, in accordance with one embodiment of the present invention. In this one embodiment for a five pound to ten pound plate, the length/height may be approximately six inches, the width may be approximately nine inches, and thickness may be approximately one inch. It should be noted that for the five pound to ten pound plate, other sizes are possible.

FIG. 3C is a front perspective of a weight plate with a cooling component integrated therein, similar to the one or single handle weight plates depicted in Figure LA and FIG. 2A, in accordance with one embodiment of the present invention. In this one embodiment for a fifteen pound to twenty pound plate, the length/height may be approximately fifteen inches, the width may be approximately seven inches, and thickness may be approximately one inch. It should be noted that for the fifteen pound to twenty pound plate, other sizes are possible.

FIG. 3D is a front perspective of a weight plate with a cooling component integrated therein, similar to the two or double handle weight plates depicted in FIG. 1B and FIG. 2B, in accordance with one embodiment of the present invention. In this one embodiment for thirty pound or heavier pound plate, the length/height may be approximately 15 inches, the width may be approximately 10 inches, and thickness may be approximately 1 inch. It should be noted that for the thirty pound to heavier plate, other sizes are possible.

FIG. 3E is a front perspective of a weight plate with a cooling component integrated therein, similar to the handless weight plates depicted in FIG. 1C and FIG. 2C, in accordance with one embodiment of the present invention. In this one embodiment for a weight plate designed to fit within the vest or a pocket of a backpack or rucksack, the plate may come in weights of 5.75 pounds and 8.75 pounds. It should be noted that for the vest plate, other weights less than, between, or greater than 5.75 pounds and 8.75 pounds are possible.

The embodiments of the present invention provides many benefits over the prior art in areas of practicality, user experience and results. The weight plate with cooling component integrated therein is designed with comfort in mind, to keep the body cool during any activity while wearing the plate in a vest, backpack, or rucksack. The ergonomic handle comfortably assists with placement and removal of the plate.

The present invention has been described in terms of specific embodiments incorporating details to facilitate the understanding of the principles of construction and operation of the invention. As such, references herein to specific embodiments and details thereof are not intended to limit the scope of the claims appended hereto. It will be apparent to those skilled in the art that modifications can be made in the embodiments chosen for illustration without departing from the spirit and scope of the invention.

We claim:

1. A weight plate with a cooling component integrated therein comprising:
a. a plate made of metal and having a hollow portion forming a cavity;
b. a cold pack positioned in the cavity; and
c. a first handle for gripping the plate.

2. The weight plate of claim 1 further comprising a fill port, which is integrally formed in the hollow portion of the plate and in fluid communication with the cold pack, wherein the fill port is sealed closed with a cap.

3. The weight plate of claim 2 wherein the metal is a non-ferrous metal.

4. The weight plate of claim 3 wherein the cold pack positioned in the cavity is encapsulated in the cavity of the plate.

5. The weight plate of claim 4 wherein the cold pack is one of an ice pack or a gel pack, containing a non-toxic blue liquid that freezer colder than water.

6. The weight plate of claim 5 further comprising a protective wrap positioned over one or more end portions of the plate, wherein the protective wrap comprises a soft neoprene type material.

7. The weight plate of claim 6 wherein the first handle for gripping is a hand held opening located between a proximal end of the cavity and a proximal end of the plate adjacent to the fill port.

8. The weight plate of claim 7 further comprising a second handle for gripping.

9. The weight plate of claim 8 wherein the second handle for gripping is a hand held opening located between a distal end of the cavity and a distal end of the plate.

10. The weight plate of claim 9 wherein the first handle and the second handle each has an elongated handle portion having rounded edges, a height of about two inches, and a width of about four and a half inches.

11. The weight plate of claim 10 wherein the plate has a weight between about 5 pounds and about 30 pounds, an overall width between 5.0 inches and 12.0 inches, an overall height or length between 5.0 inches and 20.0 inches, and an overall thickness between 0.5 inches and 1.5 inches.

12. The weight plate of claim 11 wherein the plate is carried in a rucksack, backpack, or vest against a user's body in a packing mode.

13. A weight plate with a cooling component integrated therein comprising:
a. a plate made of metal and having a hollow portion forming a cavity; and
b. a cold pack positioned in the cavity.

14. The weight plate of claim 13 further comprising a fill port integrally formed in the hollow portion of the plate and in fluid communication with the cold pack, wherein the fill port is sealed closed with a cap.

15. The weight plate of claim 14 wherein the metal is a non-ferrous metal.

16. The weight plate of claim 15 wherein the cold pack positioned in the cavity is encapsulated in the cavity of the plate.

17. The weight plate of claim 16 wherein the cold pack is an ice pack or a gel pack, containing a non-toxic blue liquid that freezes colder than water.

18. The weight plate of claim 17 further comprising a protective wrap positioned over one or more end portions or edges of the plate, wherein the protective wrap comprises a soft neoprene type material.

19. The weight plate of claim 18 wherein the plate has a weight between about 5 pounds and about 30 pounds, an overall width between 5.0 inches and 12.0 inches, an overall height or length between 5.0 inches and 20.0 inches, and an overall thickness between 0.5 inches and 1.5 inches.

20. The weight plate of claim 19 wherein the plate is carried in a rucksack, backpack, or vest against a user's body in a packing mode.
